(19) United States
(12) Patent Application Publication
(10) Pub. No.: US 2007/0073088 A1
(43) Pub. Date: Mar. 29, 2007

(54) Process for the separation, by countercurrentwise liquid-liquid extraction, of a glyoxal diacetal from a crude mixture comprising it

(75) Inventors: Olivier Simon, Cires Les Mello (FR); Francois Bleger, Trosly Breuil (FR); Alain Schouteeten, Ezanville (FR)

Correspondence Address: BROWDY AND NEIMARK, P.L.L.C.;624 NINTH STREET, NW, SUITE 300, WASHINGTON, DC, 20001-5303, US

(73) Assignee: Clariant (FRANCE), Puteaux (FR)

(21) Appl. No.: 10/557,920

(22) PCT Filed: May 18, 2004
(86) PCT No.: PCT/FR04/01224

§ 371 Date: Nov. 22, 2005

(30) Foreign Application Priority Data
May 22, 2003 (FR) 03/06168

(51) Int. Cl. C07C 43/32

(52) U.S. Cl. 568/597

(57) ABSTRACT

The invention relates to a method for separating a diacetal of the glyoxal of a raw mixture comprising said diacetal of the glyoxal and a monoacetal of the glyoxal, by means of counter-current liquid-liquid extraction.

The invention relates to a process for the separation, by countercurrentwise liquid-liquid extraction, of a glyoxal diacetal from a crude mixture comprising this diacetal as well as a glyoxal monoacetal and water.

Glyoxal acetals are synthons which are advantageous in organic synthesis (Application WO-A-02/42524) and can be used as additives (Applications EP-A-0 855 436, EP-A-0 512 501).

The most widespread method for the preparation of glyoxal diacetals is the acetalization of glyoxal by a monoalcohol R—OH with acid catalysis according to the following scheme: 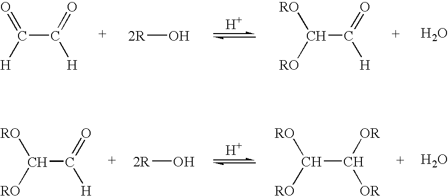
This reaction is an equilibrium reaction and results in a mixture comprising glyoxal, alcohol R—OH, water, glyoxal monoacetal and glyoxal diacetal.

The acetalization reaction is a complex reaction during which, in addition to the monoacetal and diacetal, numerous cyclic by-products and/or oligomers are formed (see, for example, J. M. Kliegman et al. in J. Org. Chem., Vol. 38 (1973), p. 556-560; A. Stambouli et al., Bull. Soc. Chimique France (1983), II, p. 33-44).

According to the prior art, the processes for the manufacture of glyoxal diacetals exhibit the disadvantage of a low yield of diacetal, that is to say of 1,1,2,2-tetraalkoxyethane, as disclosed, for example, in U.S. Pat. No. 2,360,959 (38% for tetramethoxyethane), or require the removal of the water of reaction by use of an azeotroping solvent (for example, in Patent GB 559 362), resulting in high energy and engineering costs.

Application EP 1 300 383 discloses a process for the preparation of glyoxal diacetals by reaction of 40 to 75% by weight of aqueous glyoxal with monovalent alcohols in which it is not necessary to separate, by distillation, the water formed during the acetalization reaction.

The acetalization reaction of glyoxal with an alcohol is disclosed in particular in Applications EP 0 607 772 and 0 847 976. In Application EP 0 847 976, the purification of the glyoxal diacetal co-produced during the reaction is carried out by a first distillation of the excess alcohol, followed by a distillation of an azeotrope comprising water and the glyoxal diacetal, requiring an addition of water, and finally the azeotropic distillation of the water with a third azeotroping solvent and the recovery of the anhydrous glyoxal diacetal at the bottom of the distillation column.

However, the processes disclosed in the prior art do not make it possible to carry out, easily and quantitatively, the step of separation of the glyoxal diacetal directly from the reaction mixture comprising it and the other reactants of the acetalization reaction, namely mainly glyoxal, glyoxal mono-acetal, the alcohol and water.

The technical problem to be solved thus consists in selectively separating the glyoxal diacetal from the mixture obtained on conclusion of the acetalization reaction, so as to obtain said diacetal with a high yield, this being achieved by means of a process which can be carried out equally well in batchwise mode as in continuous mode, in order to optimize the production costs.

It has now been found that such a separation can be carried out by a selective countercurrentwise liquid-liquid extraction of the glyoxal diacetal from the mixture obtained on conclusion of the acetalization reaction and of the removal of the excess alcohol, hereinafter known as “crude mixture”, using an extraction solvent which is immiscible with the reaction medium.

Advantageously, this separation process makes it possible to obtain the glyoxal diacetal with a virtually quantitative yield and in particular to recover at least 95%, indeed even at least 98%, of the amount of glyoxal diacetal present in the crude mixture.

The invention thus relates to a process for the separation of a glyoxal diacetal of formula (I)

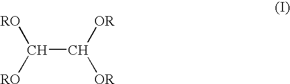

in which R represents a linear or branched C₁-C₄ alkyl group, from a mixture comprising said glyoxal diacetal and a glyoxal monoacetal of formula (II)

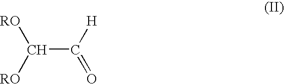

in which R is as defined above, by means of at least one step of countercurrentwise liquid-liquid extraction of said glyoxal diacetal using a solvent which is immiscible with the reaction medium chosen from ethers, alkanes and aromatic hydrocarbons, in order to obtain, on the one hand, a light phase comprising said glyoxal diacetal and, on the other hand, a heavy phase including the other constituents of the crude mixture.

The term “solvent which is immiscible with the reaction medium” is understood to mean a solvent which does not form a homogeneous mixture with the reaction medium.

In the continuation of the description, it is understood that the glyoxal monoacetal of formula (II) above can also exist in its hydrated form of following formula:

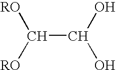

The term “linear or branched C₁-C₄ alkyl group” is understood to mean in particular a methyl, ethyl, propyl, isopropyl or butyl group.

The crude mixture from which the glyoxal diacetal of formula (I) will be separated comprises predominantly said diacetal, a monoacetal of formula (II), and water.

According to a preferred aspect of the process, the substituent R in the formulae (I) and (II) above represents a C₁-C₂ alkyl group and in particular a methyl group.

When R represents methyl, the glyoxal diacetal of formula (I) is 1,1,2,2-tetramethoxyethane (TME) and the monoacetal of formula (II) is dimethoxyethanal (DME).

In this case, the crude mixture comprises in particular, as percentages by weight, approximately 25 to 60% of TME, approximately 7 to 35% of DME and approximately 20 to 50% of water. Said mixture can also comprise, as percentages by weight, approximately 0 to 15% of glyoxal, approximately 0 to 10% of methanol and approximately 0 to 5% of impurities. The impurities are essentially composed of ethylene glycol, of acetaldehyde or of the by-products from the acetalization reaction, such as cyclic acetals, glyoxal oligomers, DME oligomers, and the like.

The choice of the extraction solvent represents a key aspect of the process.

This is because, surprisingly, it has been noticed that glyoxal diacetal, and in particular TME, has a partition behavior which is very different from that of the corresponding glyoxal monoacetal, (namely, DME in the case of TME) with respect to the solvent when the two abovementioned compounds are found in an aqueous phase. Furthermore, the partition of glyoxal diacetal, and in particular of TME, towards the solvent is very different when it is found alone in an aqueous phase and when it is found in a mixture with the corresponding glyoxal monoacetal (namely, DME in the case of TME).

Preferably, said solvent is chosen from ethers, alkanes and aromatic hydrocarbons.

Mention may be made, by way of example, of diisopropyl ether, methyl tert-butyl ether (MTBE), methyl tert-amyl ether (TAME), n-hexane, methylcyclohexane or xylene.

Solvents which are particularly advantageous for the purposes of the invention are cyclohexane, n-heptane and toluene.

The step of countercurrentwise liquid-liquid extraction can be carried out using one or more extraction columns, in series or in parallel, such as those described in particular in the reference work “Perry's Chemical Engineers' Handbook”, 7^{th} Edition, published by McGraw-Hill, 1997, chapter 15, pp. 15-28 to 15-31.

Use will preferably be made of a combination of several mixers-decanters. Use may be made, in the category of extractors operating by gravity without mechanical stirring, by way of example, of a spray tower, a packed column or a perforated plate column. Use may be made, among extractors operating by gravity with mechanical stirring, for example, of a column with a rotating stirrer or a pulsed column.

Use may also be made of a centrifugal extractor.

Use will preferably be made, for the purposes of the invention, of a column with a rotating stirrer.

According to the separation process of the invention, the crude mixture is introduced onto the liquid-liquid extraction column and is extracted using a countercurrentwise stream of extraction solvent.

The light phase comprising the glyoxal diacetal of formula (I) and the extraction solvent is collected at the column top. Advantageously, this light phase comprises at least 95%, preferably at least 98%, of the amount of glyoxal diacetal of formula (I) initially present in the crude mixture and less than 10%, preferably less than 1%, of the amount of glyoxal monoacetal of formula (II) initially present in the crude mixture.

To this end, the amount of solvent is preferably adjusted so as to extract at least 95% of the glyoxal diacetal of formula (I) while remaining as small as possible. Use will preferably be made of a solvent/crude mixture ratio by weight of between approximately 0.3/1 and 5/1, the optimum amount of solvent being adjusted according to the solvent and equipment used.

The extraction is carried out at a temperature of less than, equal to or greater than ambient temperature while remaining below the boiling point of the solvent. Preferably, the extraction is carried out at a temperature of approximately 10° C. to 60° C., preferably at ambient temperature.

The light phase obtained on conclusion of the extraction is subjected to a separation, for example by distillation under reduced pressure, on conclusion of which said glyoxal diacetal is recovered, on the one hand, and the extraction solvent is recovered, on the other hand, which extraction solvent can be recycled to the liquid-liquid extraction step.

Preferably, this distillation is carried out under reduced pressure, in particular at a pressure of the order of 200 to 300 mbar. The temperature of the distillation bottoms is preferably less than or equal to 120° C.

The heavy phase, comprising predominantly water and glyoxal monoacetal, is recovered at the column bottom and concentrated by evaporation, on conclusion of which the glyoxal monoacetal, the residual glyoxal and the residual glyoxal diacetal are recovered for the purpose of the recycling thereof.

The crude mixture is obtained on conclusion of the acetalization reaction represented above.

Use will preferably be made of an aqueous glyoxal with a concentration of between 40 and 75% by weight, preferably 60 to 70% by weight, this glyoxal being obtained by concentration under reduced pressure of commercial aqueous glyoxal, the concentration of which is generally of the order of 40%. The aqueous glyoxal will preferably be concentrated immediately before use.

The experimental conditions for the concentrating of the glyoxal and the equipment are known to a person skilled in the art and mention may be made, by way of example, of Application EP-A-1300383 or R. K. Shah and A. C. Mueller, Heat Exchange, Ullmann's Encyclopedia of Industrial Chemistry, 6^{th} edition on CD-ROM, Wiley VCH.

The alcohol R—OH/glyoxal molar ratio will preferably be between 10/1 and 50/1, preferably from 10/1 to 30/1, in particular 15/1.

Use will preferably be made of water-miscible alcohols, such as methanol, ethanol, n-propanol and isopropanol. The crude mixture comprising TME and DME is obtained by acetalization of glyoxal with methanol.

Use may equally well be made, as acid catalysts, of Lewis acids as of Brönstedt acids. Use may be made, as catalysts, of those which dissolve homogeneously in the mixture, such as, for example, sulfuric acids or organic sulfonic acids, such as methanesulfonic or p-toluenesulfonic acid. Use may also be made of heterogeneous catalysts, such as, for example, zirconium sulfate, and highly acidic, in particular sulfonic, ion exchangers. The ion-exchange resins sold under the trade names Lewatit®, BayKat® (Bayer AG, Leverkusen), Amberlite® and Amberlyst® (Röhm & Haas), and Dowex® (Dow Chemicals) are examples of appropriate ion exchangers. Lewatit®S100, Lewatit®K2431, Lewatit®K2621, Lewatit®K2629, BayKat®K2611, Amberlyst®15, Amberlyst®35 and Dowex®50 are highly acidic ion exchangers also available commercially.

Use may advantageously be made of a heterogeneous catalyst in the form of a stationary catalytic bed through which the liquid mixture comprising the alcohol and the aqueous glyoxal is introduced. The liquid mixture can pass one or more times over the stationary catalytic bed, until the desired contact time or residence time is obtained.

The temperature used for the acetalization is in principle above ambient temperature and the reaction is preferably carried out at a temperature of the order of 60° C. to 140° C., preferably from approximately 80° C. to 130° C., and more preferably of the order of 100° C. to 130° C. Preferably, the reaction is carried out at a pressure of greater than or equal to atmospheric pressure, preferably at a pressure of greater than or equal to atmospheric pressure and less than or equal to 15 bar.

The reaction of the glyoxal with alcohol is carried out until approximately the equilibrium state is reached, that is to say until the concentration of diacetal in the reaction medium is at least 70%, preferably at least 80%, more particularly at least 90% and more preferably still at least 95% of the concentration at equilibrium.

According to an advantageous aspect of the process according to the invention, the crude mixture is obtained on conclusion of an acetalization reaction carried out for a period of time of less than or equal to 1 h, preferably of less than or equal to 40 min, in particular of less than or equal to 20 min.

After having reached the reaction equilibrium, the reaction mixture is neutralized and is then distilled, preferably at atmospheric pressure with a bottom temperature of less than 110° C., to remove the excess alcohol, which is recycled in the acetalization column.

According to an advantageous aspect of the invention, the acetalization reaction, the liquid-liquid extraction step and the recovery of the various constituents of the crude mixture can be carried out continuously. In this case, the glyoxal, the glyoxal monoacetal, the alcohol R—OH and the extraction solvent are recycled.

To this end, the resulting glyoxal and the resulting glyoxal monoacetal from the concentrating of the heavy phase are reintroduced into the feed line of the column in which the acetalization is carried out. Alternatively, the heavy aqueous phase, before concentrating, can be fed directly to the evaporator used for concentrating the glyoxal.

The extraction solvent resulting from the separation of the light phase, in particular by distillation, is advantageously recovered virtually completely and is reintroduced in the feed of the liquid-liquid extraction column.

The invention is illustrated by the examples below.

### EXAMPLE 1

785 g/h of a mixture comprising, in proportions by weight, 88% of methanol, 8% of glyoxal and 4% of water, obtained by adding 686 g/h of methanol to 89 g/h of 70% glyoxal in aqueous solution, are passed, at atmospheric pressure and at a constant speed, through a column R1 which is equipped with a jacket, which has an internal diameter of 12 cm and a height of 130 cm, which is thermostatically controlled at 68±2° C. by circulation of hot water in the jacket and which comprises 13 l of cation-exchange resin comprising sulfonic groups in the acid form with an exchange capacity of 3±2 eq/l.

At the column outlet, the neutralized reaction solution, comprising, by weight, 74% of methanol, 13% of TME, 8% of water, 4.5% of DME and less than 0.5% of unconverted glyoxal, is distilled continuously at atmospheric pressure in a column C1 possessing 20 theoretical stages (DN50), with a reflux ratio of 2/1, to recover more than 99% of the methanol present, which is collected with less than 2% of water.

The recovered methanol is recycled in the feed of R1. The distillation bottoms (200±10 g/h) are continuously extracted countercurrentwise at ambient temperature by a stream of 300±10 g/h of cyclohexane in a liquid-liquid extraction column C2 of Rushton-Oldshue type with approximately thirty stirred compartments. At the column top, 400±10 g/h of light phase, which comprises more than 98% of the starting TME and less than 1% of the starting DME (cyclohexane 73.5%, TME 25.1%, DME 0.5%, water 0.9%), are collected. At the column bottom, approximately 100±10 g/h of heavy phase, which comprises approximately 58% of water, 36% of DME, 3.5% of glyoxal, 1% of TME, 0.8% of cyclohexane and 0.7% of methanol, are collected.

The light phase is continuously distilled under a pressure of 200 mbar on a column C3 (DN 50) possessing 10 theoretical stages with a reflux ratio of 1/1, to recover more than 99% of the cyclohexane present, and the DME, and to obtain, at the bottom, 100 g/h of TME with a purity of greater than 99%. The heavy extraction phase resulting from the column C2 is concentrated on an evaporator C4 at approximately 60° C.-80° C. under a pressure of 200 mbar, so as to obtain 50±5 g/h of a mixture comprising 75% of DME, 17% of water, 7% of glyoxal and 1% of TME, which mixture is mixed with 275 g/h of methanol to be recycled with the feed mixture of the column R1.

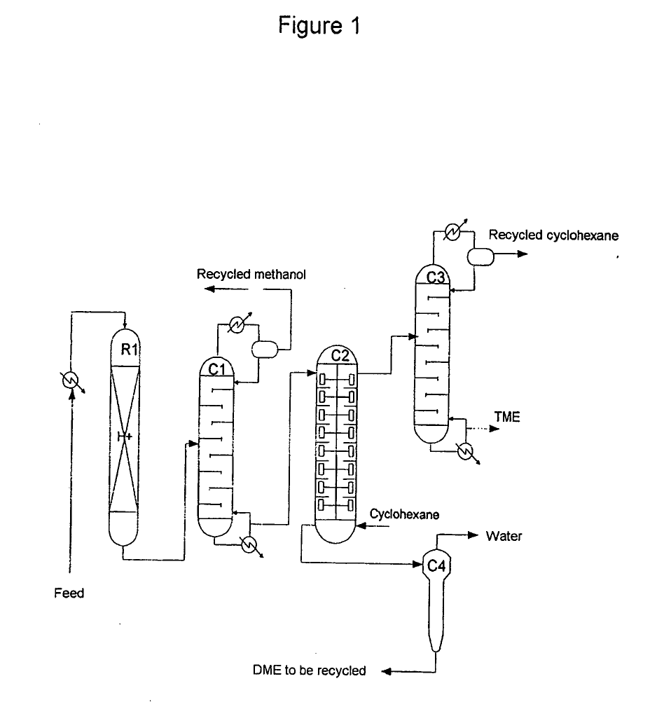

The reaction process described above is represented in FIG. 1.

### EXAMPLE 2

170 g/h of a mixture comprising 60% of water, 25% of glyoxal and 15% of DME are concentrated on a laboratory thin layer evaporator E1 under 100 mbar. The temperature of the heat-exchange fluid circulating in the jacket is adjusted to 112±2° C., so as to evaporate 85±5 g/h of water. The concentrate (85±5 g/h), comprising 50% of glyoxal, 30% of DME and 20% of water, is mixed in line with 615 g/h of methanol. This mixture is fed to a column R1 which is equipped with a jacket, which has an internal diameter of 12 cm and a height of 130 cm, which is thermostatically controlled at 68±2° C. by circulation of hot water in the jacket and which comprises 13 l of cation-exchange resin comprising sulfonic groups in the acid form with an exchange capacity of 3±2 eq/l.

At the column outlet, the neutralized reaction solution, comprising, by weight, 74.5% of methanol, 14.5% of TME, 6.5% of water, 4% of DME and less than 0.3% of unconverted glyoxal, is distilled continuously at atmospheric pressure in a column C1 possessing 20 theoretical stages (DN50), with a reflux ratio of 2/1, to recover more than 99% of the methanol present, which is collected with less than 2% of water. The recovered methanol is recycled as a mixture with the concentrate resulting from the evaporator E1.

The distillation bottoms (175±10 g/h) are continuously extracted countercurrentwise at ambient temperature by a stream of 260 g/h of cyclohexane in a liquid-liquid extraction column C2 of Rushton-Oldshue type with approximately thirty stirred compartments. At the column top, 360±10 g/h of light phase, which comprises more than 98% of the starting TME and less than 1% of the starting DME (cyclohexane 73.5%, TME 25.1%, DME 0.5% and water 0.9%), are collected. At the column bottom, approximately 70±5 g/h of heavy phase, which comprises approximately 59% of water, 38% of DME and 3% of glyoxal, are collected.

The light phase is continuously distilled under a pressure of 200 mbar on a column C3 (DN 50) possessing 10 theoretical stages with a reflux ratio of 1/1 to recover more than 99% of the cyclohexane present, and the DME, and to obtain, at the bottom, approximately 100 g/h of TME with a purity of greater than 99%.

The heavy extraction phase resulting from the column C2 is mixed with 100 g/h of a 40% aqueous glyoxal solution so as to produce a mixture comprising 60% of water, 25% of glyoxal and 15% of DME which is used as feed of R1.

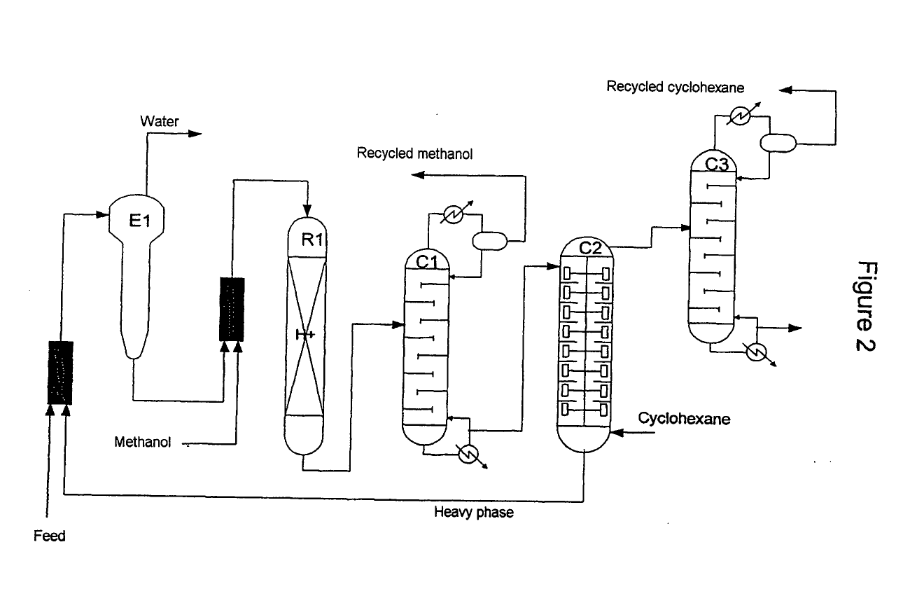

The reaction process described above is represented in FIG. 2.

### EXAMPLE 3

### Preparation of the Crude Mixture

470 g of methanol and 120 g of glyoxal as a 70.5% aqueous solution are mixed so as to obtain 590 g of a solution comprising, as percentages by weight, 79.6% of methanol, 14.4% of glyoxal and 6.0% of water, i.e. a molar ratio of 10 mol of methanol per mole of glyoxal (assayed by gas chromatography).

This solution is introduced at ambient temperature into an autoclave which has a working volume of 1 l and which is equipped with a jacket and with a stirring system, with a temperature probe and with a device for withdrawing samples.

200 g of Amberlyst® C35 resin, rinsed beforehand with methanol and dried on a Büchner funnel, are added to the medium. The autoclave is subsequently closed, its gaseous head space is purged with nitrogen and stirring is begun at 150 revolutions/min. The closed system is then heated by circulating thermostatic fluid at 120° C. in the jacket of the autoclave. In 20 min, the internal temperature reaches at least 95° C. under a pressure of approximately 3 bar. In 30 min, the internal temperature is at least 105° C. under a pressure of approximately 3.8 bar. After 40 min, the medium has reached at least 90% of the reaction equilibrium. It then comprises 1.4% of glyoxal, 15.5% of TME and 9.5% of DME.

The autoclave is cooled to 25° C. by circulating cold heat-exchange fluid in the jacket. The reaction medium is withdrawn and filtered through a Büchner funnel, so as to recover the Amberlyst® C35 resin, which can be reused.

The mixture obtained is neutralized before being purified by distillation and extraction under the conditions described in Examples 1 and 2 above, these conditions being adjusted according to the amount of reaction mixture.

### EXAMPLE 4

Study of the partition of TME between a light phase comprising cyclohexane and a heavy phase comprising water at 25° C.

50 g of an aqueous solution of TME and DME are prepared and stirred at 25° C. and atmospheric pressure in a 250 ml round-bottomed flask. 50 g of solvent are added. The medium is kept stirred for 30 min and is then transferred into a 250 ml separating funnel. After decantation, the heavy phase and the light phase are weighed and then analyzed by chromatography.

The results reported in Table 1 below are obtained by extracting 50 g of aqueous solutions of TME and DME with 50 g of cyclohexane.

TABLE 1

| | Heavy phase | | Light phase | | | |
|---|---|---|---|---|---|---|
| | DME | TME | DME | TME | Partition | Partition |
| Test | % by | % by | % by | % by | coefficient | coefficient |
| No. | weight | weight | weight | weight | TME | DME |
| 1 | 0 | 20.2 | 0 | 10.8 | 0.5 | |
| 2 | 15.3 | 23.6 | 0.15 | 26.0 | 1.1 | 0.01 |
| 3 | 32.4 | 18.6 | 0.5 | 27.6 | 1.5 | 0.02 |

The results show that, when the starting aqueous TME solution does not comprise DME (Test 1), the partition coefficient for TME (% by weight of TME in the organic phase/% by weight of TME in the aqueous phase) is equal to 0.5.

The presence of DME in the starting aqueous solution (Tests 2-3) unexpectedly increases the partition coefficient for TME. Specifically, for approximately stable amounts of TME in the heavy phase (18.6 to 23.6%), the partition coefficient for TME changes from 0.5 without DME to 1.1 with 15.3% of DME and to 1.5 with 32.4% of DME.

1. Process for the separation of a glyoxal diacetal of formula (I) 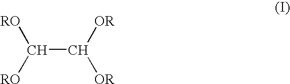
in which R represents a linear or branched C₁-C₄ alkyl group, from a crude mixture comprising said glyoxal diacetal and a glyoxal monoacetal of formula (II) 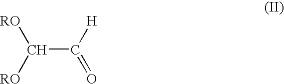
in which R is as defined above, characterized in that at least one step of countercurrentwise liquid-liquid extraction of said glyoxal diacetal is carried out using a solvent which is immiscible with the reaction medium, in order to obtain, on the one hand, a light phase comprising said glyoxal diacetal and, on the other hand, a heavy phase including the other constituents of the crude mixture.

2. Process according to claim 1, characterized in that said crude mixture comprises predominantly a glyoxal diacetal of formula (I) as defined in claim 1, a glyoxal monoacetal of formula (II) as defined in claim 1, and water.

3. Process according to claim 1 or claim 2, characterized in that the solvent is chosen from ethers, alkanes and aromatic hydrocarbons.

4. Process according to any one of claims 1 to 3, characterized in that the solvent is chosen from cyclohexane, n-heptane and toluene.

5. Process according to any one of claims 1 to 4, characterized in that the solvent/crude mixture ratio by weight is between 0.3/1 and 5/1.

6. Process according to any one of claims 1 to 5, characterized in that the extraction is carried out at a temperature of approximately 10° C. to 60° C., preferably at ambient temperature.

7. Process according to any one of claims 1 to 6, characterized in that the light phase comprising the glyoxal diacetal of formula (I) and the solvent is subjected to a separation, on conclusion of which said glyoxal diacetal is recovered.

8. Process according to claim 7, characterized in that this separation is carried out by distillation under reduced pressure.

9. Process according to either one of claims 7 and 8, characterized in that this separation is carried out at a temperature of between ambient temperature and approximately 120° C.

10. Process according to any one of claims 1 to 9, characterized in that the solvent is recycled to the liquid-liquid extraction step.

11. Process according to any one of claims 1 to 10, characterized in that the crude mixture is obtained by an acetalization reaction of 40 to 75% by weight aqueous glyoxal with an alcohol of formula R—OH in which R is as defined in claim 1, the R—OH/glyoxal molar ratio being between 10/1 and 50/1, preferably 10/1 to 30/1, in the presence of an acid catalyst, followed by the distillation of the reaction mixture obtained in order to remove the excess alcohol R—OH.

12. Process according to any one of claims 1 to 11, characterized in that, in the formulae (I) and (II), R is a C₁-C₂ alkyl group.

13. Process according to claim 12, characterized in that R is a methyl group.

14. Process according to any one of claims 1 to 13, characterized in that the alcohol is methanol.

15. Process according to any one of claims 1 to 14, characterized in that the crude mixture comprises predominantly 1,1,2,2-tetramethoxy-ethane (TME), dimethoxyethanal (DME) and water.

16. Process according to any one of claims 1 to 15, characterized in that said mixture comprises, as percentages by weight, approximately 25 to 60% of TME, approximately 7 to 35% of DME and approximately 20 to 50% of water.

17. Process according to any one of claims 1 to 16, characterized in that said mixture also comprises, as percentages by weight, approximately 0 to 15% of glyoxal, approximately 0 to 10% of methanol and approximately 0 to 5% of impurities.

18. Process according to any one of claims 11 to 17, characterized in that the glyoxal used in the acetalization reaction is concentrated to approximately 60 to 70%.

19. Process according to claim 18, characterized in that the glyoxal is concentrated from an aqueous solution.

20. Process according to any one of claims 11 to 19, characterized in that the acetalization reaction is carried out for a period of time of less than or equal to 1 h, preferably of less than or equal to 40 min.

21. Process according to claim 20, characterized in that the period of time of the reaction is less than or equal to 20 min.

22. Process according to one of claims 11 to 21, characterized in that the acetalization reaction is carried out at a temperature of the order of 60° C. to 140° C., preferably approximately 80° C. to 130° C.

23. Process according to claim 22, characterized in that the temperature is of the order of 100 to 130° C.

24. Process according to one of claims 11 to 23, characterized in that the acetalization reaction is carried out at a pressure of greater than or equal to atmospheric pressure.

25. Process according to claim 24, characterized in that the pressure is less than or equal to 15 bar.

26. Process according to any one of claims 1 to 25, characterized in that the acetalization reaction, the liquid-liquid extraction step and the recovery of the various constituents of the crude mixture are carried out continuously, the glyoxal, the glyoxal monoacetal, the alcohol R—OH and the extraction solvent being recycled.
